(19) United States
(12) Patent Application Publication
(10) Pub. No.: US 2010/0010591 A1
(43) Pub. Date: Jan. 14, 2010

(54) NON-INVASIC THERAPEUTIC TREATMENT DEVICE

(75) Inventor: Steven J. Daffer, Edina, MN (US)

Correspondence Address: WESTMAN CHAMPLIN & KELLY, P.A., SUITE 1400, 900 SECOND AVENUE SOUTH, MINNEAPOLIS, MN, 55402, US

(73) Assignee: Sybaritic, Inc., Bloomington, MN (US)

(21) Appl. No.: 12/437,142

(22) Filed: May 7, 2009

Related U.S. Application Data
(60) Provisional application No. 61/051,209, filed on May 7, 2008.

(51) Int. Cl. A61N 5/06; A61M 35/00

(52) U.S. Cl. 607/88; 604/289

(57) ABSTRACT

A non-invasive therapeutic device includes a housing comprising a cavity and a bottom surface. A plurality of light emitting arrays are positioned within the bottom surface. At least one fan positioned within the cavity to provide at least cooling to the patient. The device includes a controller positioned on the housing for controlling the plurality of light emitting arrays and the at least one fan such that a phototherapy treatment can be applied to a selected region of a patient.

### CROSS REFERENCE TO RELATED APPLICATION(S)

This application claims the benefit of U.S. Provisional Application Ser. No. 61/051,209, which was filed on May 7, 2008, the contents of which are incorporated by reference in its entirety.

### FIELD OF THE INVENTION

The present invention relates to an apparatus for improving the appearance of a person's skin through non-invasive means. The present invention utilizes an apparatus having numerous modalities of non-invasive treatments to rejuvenate and repair a patient's skin including phototherapy light treatment and elevated oxygen concentration treatments.

### BACKGROUND OF THE INVENTION

Many people strive to delay the aging process. As a person ages, the skin begins to lose its elasticity, become wrinkled and may develop spots or blemishes. Some people would go to great lengths to look younger including subjecting themselves to invasive procedures. Some invasive procedures include a face lift where skin is removed and tightened around the face as well having spots surgically removed from the skin.

While the invasive procedures can be effective in obtaining a younger looking appearance, the patient experienced days of discomfort/pain after the procedure. Also, any time a person is subjected to a surgical procedure where the person is anesthetized, such as a face-lift, there are health risks. Further, when a surgical procedure is performed the results are not guaranteed and the patient is subjected to the possibility of infection.

### SUMMARY OF THE INVENTION

The present invention relates to a non-invasive treatment device that is utilized on a patient's skin. The device is positioned proximate a portion of a person's skin and delivers at least one therapeutic treatment to the skin. A non-exhaustive list of therapeutic treatments includes phototherapy where the skin is subject to light waves, subjecting the skin to a therapeutic gas and aromatherapy. Along with the therapeutic treatments, a transparent sheet that is positioned over a selected area of skin or mask which can be placed over the patient's face can be utilized where the sheet or mask includes photo-sensitive ingredients which are activated by the lights utilized in the phototherapy.

### BRIEF DESCRIPTION OF THE DRAWINGS

FIG. 1 is a perspective view of a non-invasive skin therapy device.

FIG. 2 is a perspective view of the non-invasive skin therapy device being utilized on a patient in a prone position.

FIG. 3 is a partial perspective view of the piping for introducing a therapeutic gas onto or near a patient's skin.

FIG. 4 is a perspective view of the interior of the housing of the non-invasive skin therapy device.

### DETAILED DESCRIPTION

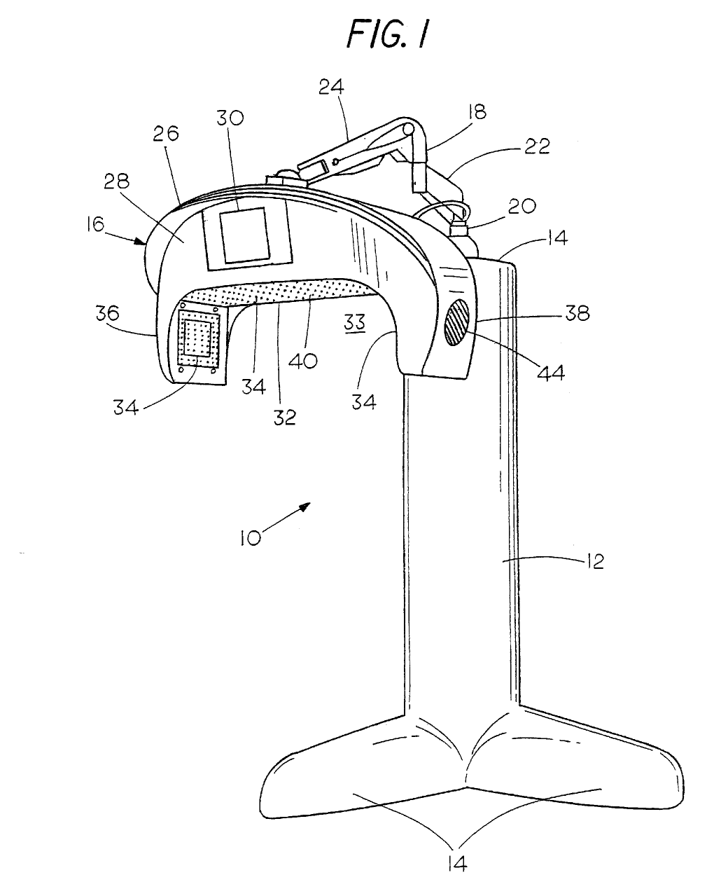

A device for therapeutically treating a patient's skin with non-invasive treatments is generally indicated at 10 in FIG. 1. The device 10 includes a stand 12 that is supported by a plurality of feet 14 that engage a surface or floor. The plurality of feet provide a base which supports the device 10 in a substantially upright configuration. A non-invasive skin therapy unit 16 is pivotally attached to a top end 14 of the stand 12.

The non-invasive skin therapeutic unit 16 is pivotally attached to the top end 14 of the stand with a top pivot 18 and a bottom pivot 20 where the top and bottom pivots 18, 20. The top and bottom pivots 18 and 20 are connected via an intermediate member 22. A top member 24 is pivotally attached to the top pivot 22 and a bracket attached to a housing 26 of the non-invasive skin therapy unit 16. Utilizing multiple pivots allows the non-invasive skin therapy unit 16 to be placed in a wide range of positions in three dimensions.

The housing 26 includes an interior opening for containing the therapeutic modalities as well as the electronics that control the output of the device. A front wall 28 of the unit 16 includes a touch screen 30 which allows a care provider to program the type of treatment for the patient including the intensity, time and modalities of treatment being delivered to the patient's skin. However, other control devices besides a touch screen are also contemplated including a personal computer that is communication with the device.

The housing 26 includes a substantially U-shaped interior bottom wall 32 that includes a plurality of light emitting diode (LED) arrays 34. The LED arrays 34 are contained within left and right side surfaces 36 and 38, respectively, as well as a top surface 40, which connects the side surfaces 36 and 38. While a unit with a substantially U-shaped interior bottom wall 32 is illustrated, other configurations of the unit and the bottom wall are also contemplated including an arcuate bottom wall and a V-shaped bottom wall and a polygonal bottom wall.

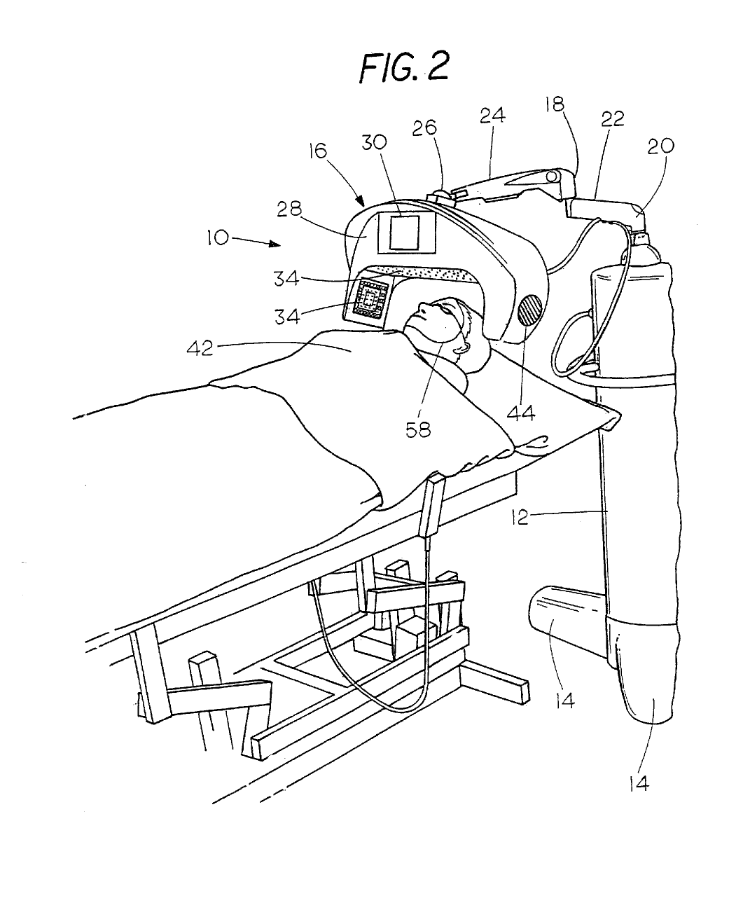

Referring to FIG. 2, a patient 42 is depicted positioned within a U-shaped cavity 33 where the patient's face is being non-invasively treated. However, other regions of the body can also be non-invasively treated with the device including but not limited to the breast, abdomen, buttocks, thighs, etc. The areas of the body are then subjected to the selected modalities of therapy.

Once the patient is situated under the bottom wall 32, the LED arrays 34 can be manipulated and controlled to provide selected colors to provide a selected treatment, such as red light having a wavelength of 630 nanometers and a yellow light having a wavelength of 590 nanometers. Also, a combination of red and yellow can be used to provide a selected treatment. Other colors, including a blue wavelength 405-450 nm that provide therapeutic treatments to the skin are also contemplated.

The patient can also utilize a transparent gel mask 58 or sheet which can be placed over the patient's face or other region of the body. The mask 58 or sheet can include photo-sensitive ingredients which activate when subjected to a selected wavelength of light. The wavelength typically corresponds to the phototherapy treatment. Typical photo-sensitive therapeutic ingredients include, but are not limited to, a microgel firming agent, a skin-whitening agent, an acne-clearance agent, a cellulite reduction agent, a skin firming and/or tightening agent and a deep hydration agent. The combination of the photo-sensitive ingredient therapy along with the phototherapy provided by the LED light arrays provide for enhancement of the active ingredients and a deeper penetration into the skin which improves the effectiveness of the treatment that cannot be accomplished when the photo-sensitive ingredient and phototherapy are applied to the patient in sequence.

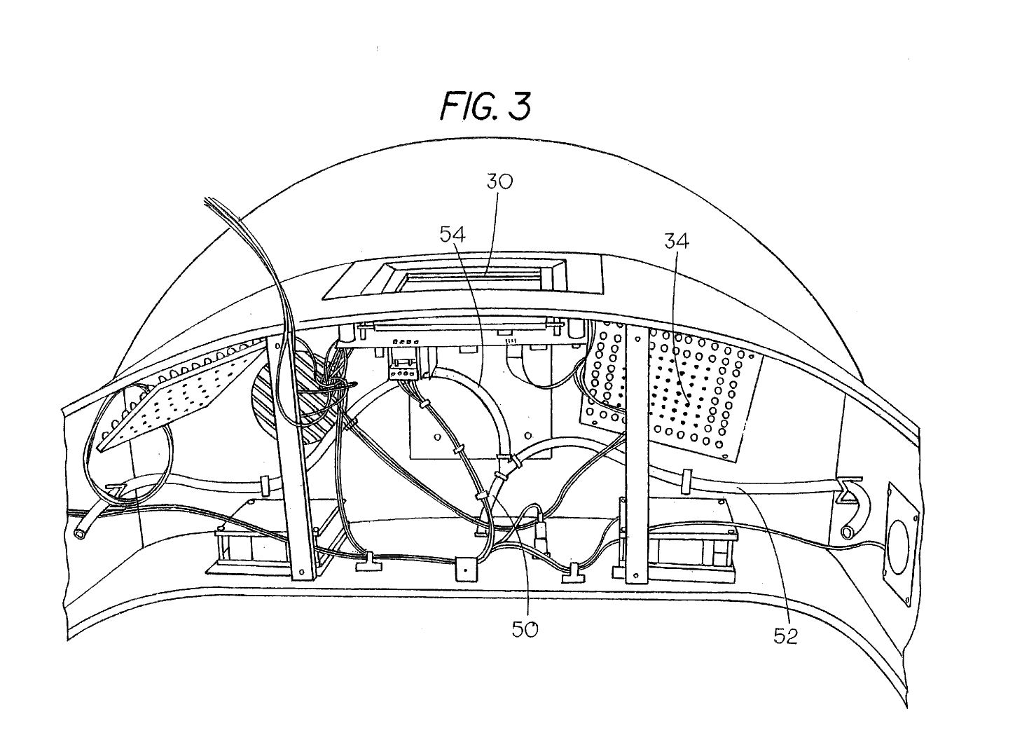

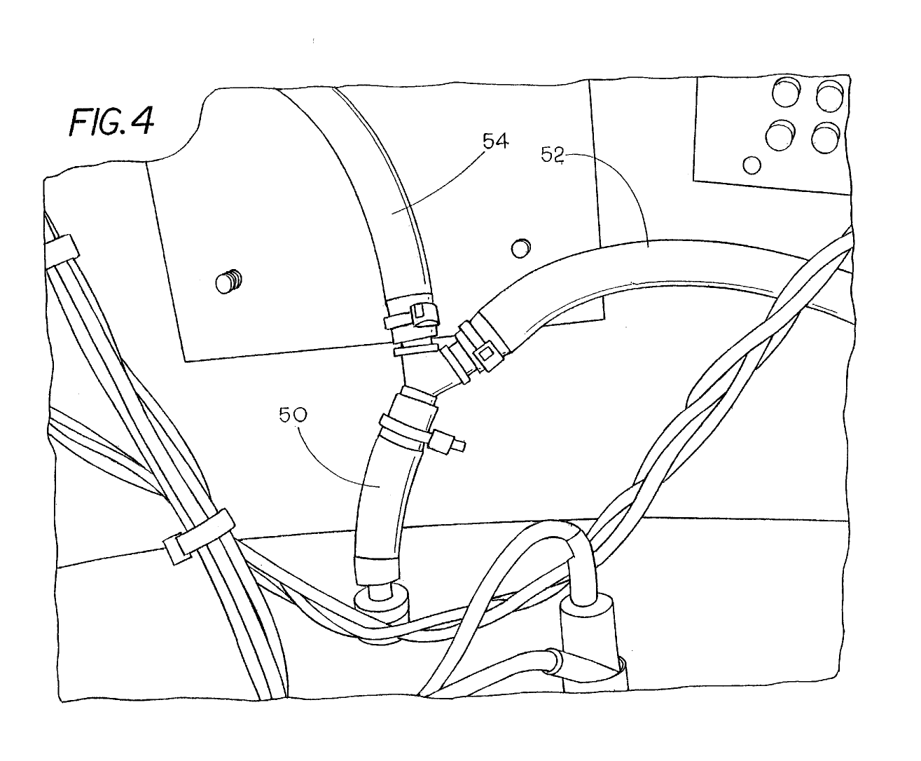

The phototherapy causes the skin of the patient to begin to heat because the skin is absorbing light energy. Therefore, the unit 16 includes left and right fans 44 (one of which is shown), which force air onto the patient's face to keep the patient's face cool. Along with the air that is forced into the patient's face or other body region for cooling, a therapeutic gas can also be applied to the patient's face via tubing 50 as best illustrated in FIGS. 3 and 4. The gas delivery system 50 supplies the therapeutic gas from a common tube to left and right tubes 52, 54 that branch away from the main tube 50 that typically is attached to a supply source such as a portable tank or piping that supplies the therapeutic gas throughout a facility. The left and right tubes 52, 54 discharge proximate the left and right fans 44 (one of which is shown) to provide the therapeutic gas that can provided ionization or germicidal cleansing to the region being treated. Along with the therapeutic gas, a mist or scent may also be added to the gas such as to provide aromatherapy, a medicinal spray such as Vitamin C, a therapeutic respiratory medicinal mist, or any other topical treatment.

Optionally, a shroud can be placed over the front and back surfaces of the unit 16 to provide an enclosure such that the concentration of the therapeutic gas, such as oxygen, can be increased above that of the ambient level. Also, the device can optionally include a sensor such as an oxygen sensor to monitor the concentration of the therapeutic agent in the enclosure. Also, optionally, the unit 26 may include a controller (not shown) to control the concentration of the therapeutic agent, such as oxygen, being supplied through both the left and right tubes 52, 54 to control the concentration of the therapeutic agent within the enclosure to a preset set point utilizing a feedback control system where the sensor provides a signal to adjust the controller to provide the therapeutic agent at the selected concentration.

The touch screen 30 or other control device can be manually operated to provide a selected intensity of the lights for a selected amount of time through manual operation. The touch screen 30 can also be utilized to run a preset program that provides a multiple modality treatment at one time as well as vary and randomize the amount of treatment and the intensity of the treatment and the pulsation or continuous application of the light exposure over periods of time through a treatment cycle.

By randomizing the duration and intensity of the lights upon the skin, it has been discovered that the skin better responds to the treatment as it is not preconditioned to anticipate receiving a selected treatment. This effect is similar to the effect on muscles that perform various exercises instead of consistently repeating one exercise. While there is benefits to exercising, it is found that by varying the exercise, greater gains are obtained to the fitness of the muscle. The same holds true by varying the intensity and duration of the light exposure to the skin. Also, there is a synergetic effect by utilizing both phototherapy, the photo-sensitive ingredients in the mask and the therapeutic gases. Any combination of the two modalities, including photo-sensitive application of therapy and/or therapeutic gases increases the effectiveness of the treatment on the skin as compared to providing the same treatment for the same amount of time in sequence.

It has been discovered that utilizing a red light accelerates the healing of wounds in a patient. Similarly, it has been discovered that utilizing increased oxygen levels also reduces bacteria and accelerates healing of a patient's wounds. Therefore, the inventive device need not only be utilized for purely cosmetic reasons of increasing the smoothness and tone of the skin. Rather the inventive device can also be utilized for therapeutic reasons, such as reducing the amount of time needed to heal the skin.

Although the present invention has been described with reference to preferred embodiments, workers skilled in the art will recognize that changes may be made in form and detail without departing from the spirit and scope of the invention.

What is claimed is:

1. A non-invasive therapeutic device comprising:
a housing comprising a cavity and a bottom surface;
a plurality of light emitting arrays positioned within the bottom surface;
at least one fan positioned within the cavity; and
a controller positioned on the housing for controlling the plurality of light emitting arrays and the at least one fan such that a phototherapy treatment can be applied to a selected region of a patient.

2. The device of claim 1 and wherein the bottom surface comprises a substantially U-shaped having a first side wall, a second side wall and a top portion connecting the first and second side walls.

3. The device of claim 2 and wherein the plurality of light emitting arrays comprises three light emitting arrays wherein a first light emitting array is positioned within the first side wall, a second light emitting array positioned within the second sidewall and a third light emitting array positioned within the top portion.

4. The device of claim 3 and wherein the controller controls the light wavelength, the intensity of the light and the duration of the light being utilized as the phototherapy treatment emitted from the plurality of light emitting arrays.

5. The device of claim 1 and wherein the controller controls the light wavelength, the intensity of the light and the duration of the light being utilized as the phototherapy treatment emitted from the plurality of light emitting arrays.

6. The device of claim 1 and wherein the plurality of light emitting arrays comprise light emitting diodes (LED).

7. The device of claim 1 and further comprising a gas delivery system that supplies a therapeutic gas or vapor proximate the at least one fan such that the fan directs the therapeutic gas or vapor to the selected region of the patient.

8. The device of claim 7 and wherein the therapeutic gas or mist comprises oxygen.

9. The device of claim 7 and where the therapeutic gas or mist comprises an aromatherapy scent.

10. The device of claim 7 and where the therapeutic gas or mist comprises a medicinal spray.

11. The device of claim 7 and where the therapeutic gas or mist comprises medicinal mist.

12. The device of claim 1 and wherein the selected region comprises the face of the patient.

13. The device of claim 1 and further comprising a transparent mask or a transparent sheet positioned on the selected region of the body wherein the mask or sheet contains a photo-sensitive ingredient that is activated by the plurality of light emitting arrays and provides a desired treatment to the patient.

14. The device of claim 13 and wherein the photo-sensitive ingredient comprises a microgel firming agent, a skin-whitening agent, an acne-clearance agent, a cellulite reduction agent, a skin firming and/or tightening agent and a deep hydration agent.

15. A non invasive kit for treating a patient's face, the kit comprising:
a non-invasive therapeutic device comprising:
a housing comprising a cavity and a bottom surface;
a plurality of light emitting arrays positioned within the bottom surface;
at least one fan positioned within the cavity; and
a controller positioned on the housing for controlling the plurality of light emitting arrays and the at least one fan such that a phototherapy treatment can be applied to a selected region of a patient; and
a transparent mask positioned on the patient's face wherein the mask contains at least one photo-sensitive ingredient that is activated by the plurality of light emitting arrays and provides a desired treatment to the patient wherein the combination of the phototherapy treatment along with the photo-sensitive ingredient is more effective at treating the patients face in combination than when the phototherapy treatment and photo-sensitive ingredient are applied at different times.

16. The kit of claim 15 and wherein the photo-sensitive ingredient comprises a microgel firming agent, a skin-whitening agent, an acne-clearance agent, a cellulite reduction agent, a skin firming and/or tightening agent and a deep hydration agent.

17. The device of claim 15 and wherein the bottom surface comprises a substantially U-shaped having a first side wall, a second side wall and a top portion connecting the first and second side walls.

18. The device of claim 17 and wherein the plurality of light emitting arrays comprises three light emitting arrays wherein a first light emitting array is positioned within the first side wall, a second light emitting array positioned within the second sidewall and a third light emitting array positioned within the top portion.

19. The device of claim 18 and wherein the controller controls the light wavelength, the intensity of the light and the duration of the light being utilized as the phototherapy treatment emitted from the plurality of light emitting arrays.

20. The device of claim 15 and wherein the controller controls the light wavelength, the intensity of the light and the duration of the light being utilized as the phototherapy treatment emitted from the plurality of light emitting arrays.
